(19) United States
(12) Patent Application Publication
(10) Pub. No.: US 2005/0267496 A1
(43) Pub. Date: Dec. 1, 2005

(54) Pressure powered anastomotic system

(75) Inventors: Amir Loshakove, Moshav-Bazra (IL); Nimrod Kaufmann, Givataim (IL); Ido Kilemnik, Herzelia (IL); Gil Hefer, Kfar-Saba (IL)

Correspondence Address: William H. Dippert, Esq.;c/o Reed Smith LLP, 29th Floor, 599 Lexington Avenue, New York, NY, 10022-7650, US

(73) Assignee: By-Pass, Inc., Orangeburg, NY (US)

(21) Appl. No.: 10/951,437

(22) Filed: Sep. 27, 2004

Related U.S. Application Data
(63) Continuation-in-part of application No. PCT/IL04/00311, filed on Apr. 4, 2004.
(63) Continuation-in-part of application No. PCT/IL03/00959, filed on Nov. 13, 2003.
(60) Provisional application No. 60/492,998, filed on Aug. 7, 2003.
(60) Provisional application No. 60/561,092, filed on Apr. 8, 2004.
(60) Provisional application No. 60/561,091, filed on Apr. 8, 2004.
(60) Provisional application No. 60/518,677, filed on Nov. 12, 2003.
(60) Provisional application No. 60/505,946, filed on Sep. 25, 2003.

(51) Int. Cl. A61B 17/08

(52) U.S. Cl. 606/153

(57) ABSTRACT

A fluid-based anastomosis system, including a body, a fluid power source, an actuator powered by said source and at least two elements coupled to said actuator and adapted to perform at least two different relative motions, responsive to serial powering of said actuator.

### RELATED APPLICATIONS

This application is a continuation-in-part of PCT Application No. PCT/IL2004/000311, filed on Apr. 4, 2004, which designates the US and which claims the benefit under 35 U.S.C. §119(e) of U.S. Provisional Application No. 60/492,998, filed on Aug. 7, 2003. This application is also a continuation-in-part of PCT Application No. PCT/IL03/00959, filed on Nov. 13, 2003, published as WO 2004/043216, which designates the US. This application also claims the benefit under 35 U.S.C. §119(e) of U.S. Provisional No. 60/561,092, filed on Apr. 8, 2004, U.S. Provisional No. 60/561,091, filed on Apr. 8, 2004, U.S. Provisional No. 60/518,677, filed on Nov. 12, 2003, and U.S. Provisional No. 60/505,946, filed on Sep. 25, 2003. The disclosures of the above applications are incorporated herein by reference.

### BACKGROUND OF THE INVENTION

Two blood vessels can be connected to form an anastomotic connection in many methods, including, for example, using surgical clips, using sutures, and using anastomotic connectors, for example as provided by Kaster in U.S. Pat. No. 5,234,447, the disclosure of which is incorporated herein by reference.

In the Kaster system and in other systems known in the art, the source of power used in attaching the anastomotic connector is direct mechanical force applied by a human, in some cases using a means of mechanical gain.

PCT publication WO 99/62415, the disclosure of which is incorporated herein by reference, suggests using a pneumatic or hydraulic pressure build up to shoot a blood vessel penetration tip through a blood vessel wall.

### SUMMARY OF THE INVENTION

An aspect of some embodiments of the invention relates to powering an anastomosis delivery system using a pressure source. In an exemplary embodiment of the invention, a hydraulic pressure source is used. Optionally, the hydraulic pressure source is volume controlled, rather than pressure controlled, with different operational states of the delivery system being set by changing a target volume of the hydraulic system (e.g., both the source and the delivery system). In an alternative embodiment, a pneumatic pressure source is used. Optionally, a mechanical means is provided to stop the progress of the anastomosis delivery at stages, so that a user intervention is required to allow the process to progress to a next stage.

In an exemplary embodiment of the invention, the power source is used to tear off a portion of a connector, after the connector is inserted into place. Optionally, the pressure source is used to both advance and retract portions of the delivery system relative to a target blood vessel. In an exemplary embodiment of the invention, the delivery system completes an anastomosis by the delivery of a connector. In some embodiments, the connector, at least after deployment, comprises a plurality of individual clips.

In some embodiments, the pressure source is used to power all the actions of the delivery, including, for example, approximation, incision opening, connector locking and tearing. Alternatively, fewer than all actions are powered by the pressure source. In some embodiments of the invention, a combined mechanical/fluid delivery system is used. For example, a mechanical system can be used to control an order of stages and/or the initiation and/or availability of stages, with the pressure providing the power.

Optionally, advancing of the anastomosis process is provided by manipulating the pressure source, for example, increasing pressure levels.

In an exemplary embodiment of the invention, a floating cylinder-piston mechanism is provided in which either one of the cylinder and piston can move (when a chamber in the mechanism is expanded), up to a limit defined by a body of the system. This allows both forward and reverse motion to be provided, in stages, with a single cylinder-piston mechanism. Optionally, one or more pins, ball-locks, friction and/or other mechanical means is used to direct the motion.

In an exemplary embodiment of the invention, a user applies a small force (e.g., pressing a soft button) to control the application of a larger force, which larger force provides a deployment of an anastomosis connector. Optionally, the larger force is separately provided by the user. Alternatively, it is machine provided.

In an exemplary embodiment of the invention, a user provides control of the activation of various stages in a deployment. Optionally, such control is provided by stepped increases in pressure and/or changes in volume of a fluid source. Optionally, such control comprises positional control, in which a user directs the system to achieve a particular position, rather than a force control, in which a user decides what force to apply. Optionally, the control is fine enough to allow tearing of individual legs of a connector in a serial manner, so that not all legs are torn simultaneously.

An aspect of some embodiments of the invention relates to a pressure-powered delivery system adapted to fit an anastomosis delivery capsule (i.e., a distal head of a delivery system), which also fits a manually deploying delivery system. Optionally, a single pressure powered handle may be used with various types of capsules, which can be replaced in the handle. The capsules can be used with a mechanically powered system as well.

An aspect of some embodiments of the invention relates to a pressure-powered delivery capsule adapted to be connected to a pressure source and deploy (e.g., position, lock and/or tear) an anastomosis connector when pressure is applied by said source. Optionally, the deployment is in distinct stages.

An aspect of some embodiments of the invention relates to a fluid based delivery system in an expanding chamber is adjacent a graft, for example, surrounding the graft to be delivered, behind the graft and/or is to one side thereof. Optionally, the chamber is within 4 cm, 3 cm, 2 cm, 1 cm or less from the graft.

An aspect of some embodiments of the invention relates to sealing element attached to a graft delivery system, which sealing element gently squeezes a graft to prevent blood flow therethrough. Optionally, the sealing element also prevents flow of blood through and out of the delivery system.

There is thus provided in an exemplary embodiment of the invention, a fluid-based anastomosis system, comprising:
a body;
a fluid power source;
an actuator powered by said source; and
at least two elements coupled to said actuator and adapted to perform at least two different relative motions, responsive to serial powering of said actuator. Optionally, at least one of said motions comprises retraction of at least one of said elements relative to said body. Alternatively or additionally, at least one of said motions comprises advancing of at least one of said elements away from said body. Alternatively or additionally, a same actuator both extends and retracts a same element relative to said body, using a positive applied pressure. Alternatively or additionally, said actuator comprises a piston-chamber mechanism that floats relative to said body. Alternatively or additionally, said actuator selectively interlocks with said body. Alternatively or additionally, at least one of said motions comprises rotation of at least one of said elements relative to said body.

In an exemplary embodiment of the invention, said system comprises a triggering mechanism which selectively unlocks at least one of said elements to move.

Optionally, said motions are controlled by a level of pressure provided by said source. Alternatively or additionally, said motions are controlled by volume provided by said source.

In an exemplary embodiment of the invention, said actuator is a hydraulic actuator. Alternatively or additionally, said actuator is a pneumatic actuator.

In an exemplary embodiment of the invention, said body is splittable axially. Alternatively or additionally, said system comprises a stop adapted to stop a movement at a predefined amount of motion.

In an exemplary embodiment of the invention, said system comprises a stop adapted to stop at least one of said movements at a predefined amount of motion.

Optionally, at least one of said elements comprises a punch. Alternatively or additionally, at least one of said elements comprises an anastomosis connector. Optionally, said motions comprise advancing and retracting said connector. Alternatively or additionally, said motions comprise tearing said connector.

In an exemplary embodiment of the invention, body is mounted on a flexible tube. Alternatively or additionally, said body is adapted to be held by hand.

There is also provided in accordance with an exemplary embodiment of the invention, a method of performing an anastomosis delivery process, comprising;
providing an anastomosis related tool at a blood vessel;
applying fluid pressure to actuate said tool; and
further applying fluid pressure to differently actuate said tool.

There is also provided in accordance with an exemplary embodiment of the invention, a connector delivery system, comprising:
a fluid power source;
an actuator powered by said source; and
at least two elements coupled to an anastomosis connector, which actuator moves one element relative to the other element to deploy the connector. Optionally, said actuator moves one element relative to the other element in at least two different movements, such that one movement retracts the connector relative to one element and the other movement releases the connector. Alternatively or additionally, the connector is released by being torn. Alternatively or additionally, that the pressure source controls the two movements.

There is also provided in accordance with an exemplary embodiment of the invention, a fluid-based anastomosis system, comprising:
a body;
a fluid power source;
an actuator powered by said source; and
at least one tool adapted to be retracted towards said body when said actuator is powered.

There is also provided in accordance with an exemplary embodiment of the invention,
a fluid powered anastomotic system, comprising:
a fluid power source;
a handle attached to said source;
an actuator inside said handle; and
a receptacle defined in said body and adapted to receive a mechanically actuated capsule, said actuator adapted to engage a body inserted in said receptacle.

There is also provided in accordance with an exemplary embodiment of the invention, a fluid powered anastomotic system, comprising:
a body adapted to receive a graft vessel; and
an expanding fluid receiving chamber adjacent the graft. Optionally, said chamber surrounds said graft. Alternatively or additionally, said chamber is to a side of said graft.

There is also provided in accordance with an exemplary embodiment of the invention, a graft delivery system comprising:
a body adapted to receive said graft; and
a flexible apertured element adapted to apply pressure to said graft and prevent blood flow therethrough. Optionally, said element prevents flow out of said body.

### BRIEF DESCRIPTION OF THE FIGURES

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1 is a schematic diagram of a pressure-powered anastomosis connector delivery system, in accordance with an exemplary embodiment of the invention;

FIGS. 2A and 2B show an outside view and a cross-sectional view of a powered handle, in a first deployment stage, in accordance with an exemplary embodiment of the invention;

FIG. 2C shows a capsule inserted into a powered handle, in accordance with an exemplary embodiment of the invention;

FIGS. 3A and 3B show an outside view and a cross-sectional view of a powered handle, in a second deployment stage, in accordance with an exemplary embodiment of the invention;

FIGS. 4A, 4B and 4C show an outside view and two cross-sectional views of a powered capsule, in a first deployment stage, in accordance with an exemplary embodiment of the invention;

FIGS. 5A, 5B and 5C show an outside view and two cross-sectional views of a powered capsule, in a second deployment stage, in accordance with an exemplary embodiment of the invention;

FIGS. 6A, 6B and 6C show an outside view and two cross-sectional views of a powered capsule, in a third deployment stage, in accordance with an exemplary embodiment of the invention;

FIGS. 7A-7F are cross-sectional views of an alternative embodiment of a powered capsule, in accordance with an exemplary embodiment of the invention;

FIGS. 8A and 8B show a complete powered capsule system before and after deployment, in accordance with an exemplary embodiment of the invention;

FIG. 9 shows a hydraulic powered punching capsule, in accordance with an exemplary embodiment of the invention; and

FIG. 10 shows a hollow hydraulic capsule, in accordance with an exemplary embodiment of the invention.

### DETAILED DESCRIPTION OF EXEMPLARY EMBODIMENTS

Overview

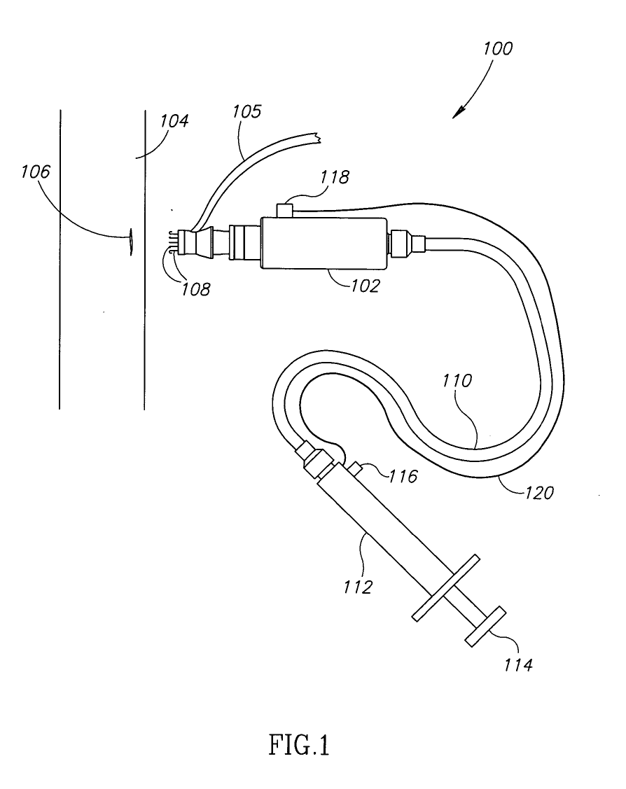

FIG. 1 is a schematic diagram of a pressure-powered anastomosis connector delivery system 100, in accordance with an exemplary embodiment of the invention. A graft vessel 105 is to be connected to a target vessel 104 at an aperture 106 thereof. In an exemplary embodiment of the invention, an anastomotic connector 108 is held in a delivery module 102, which is attached by a tube 110 to a hydraulic (or other) pressure source 112. Various exemplary types of delivery modules are described below. In an exemplary embodiment of the invention, pressure source 112 is a syringe with a movable piston 114. In an exemplary embodiment of the invention, piston 114 is advanced by rotation, possibly allowing finer user control. Alternatively or additionally, a motorized pressure source is used, in which a pump provides the pressure. Alternatively, pressure is provided from standard hospital systems, for example, a source of CO₂. FIG. 1 further shows optional control elements 120, 118 and 116, which are described below.

In an exemplary embodiment of the invention, connector 108 is of a tearing type described in PCT publication WO 00/56226, for example, in which deployment of the connector includes a process of tearing the connector off of one or more extensions. In an exemplary embodiment of the invention, the pressure from source 112 is used to first close the connection between graft 105 and target vessel 104 by retracting the legs of connector 108 relative to delivery system 100 (or advancing the delivery system 100 towards the blood vessel). Then, the pressure from pressure source 112 is used to tear the connector 108 off of delivery module 102. As will be noted below, a pressure-based system may also be used for non-tearing connectors, for example connectors wherein a last step of deployment is release of backwards legs, rather than tearing the connector off of the delivery system.

An advantage of some implementations of the invention is that the large forces involved in such tearing do not interfere with a holding of delivery module 102 in position. In some embodiments, one or more of the following advantages is provided: low recoil, smooth operation, control of various stages of operation, optional pausing in intermediate steps and/or between tearing of individual legs, suitability for keyhole surgery and suitability for robotic surgery.

Powered Handle

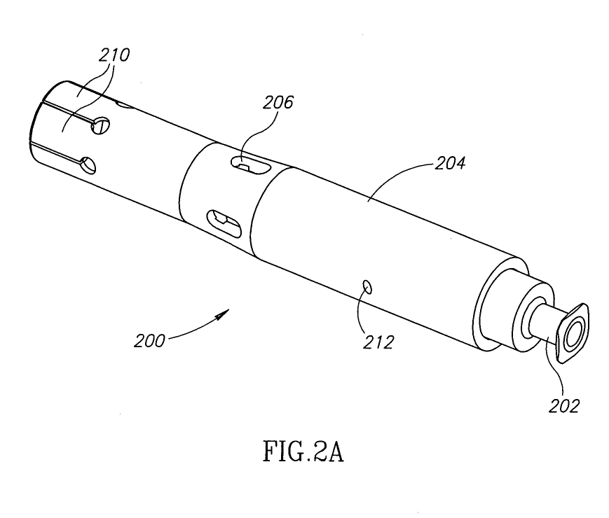

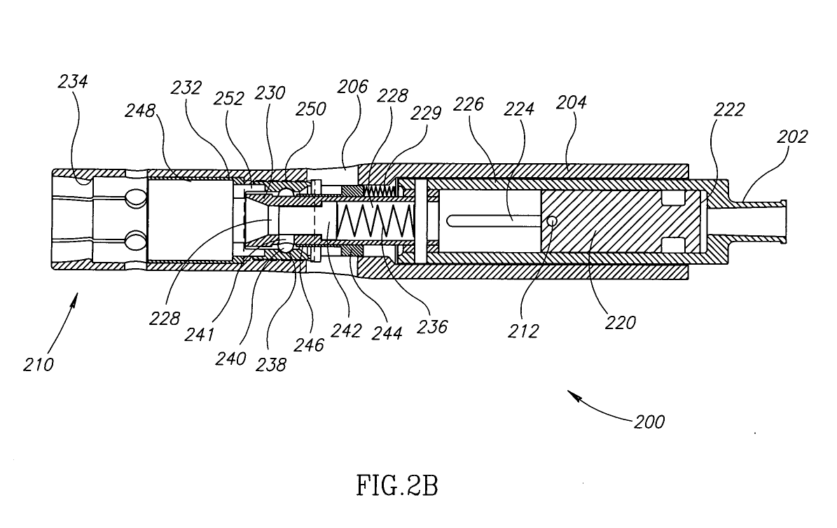

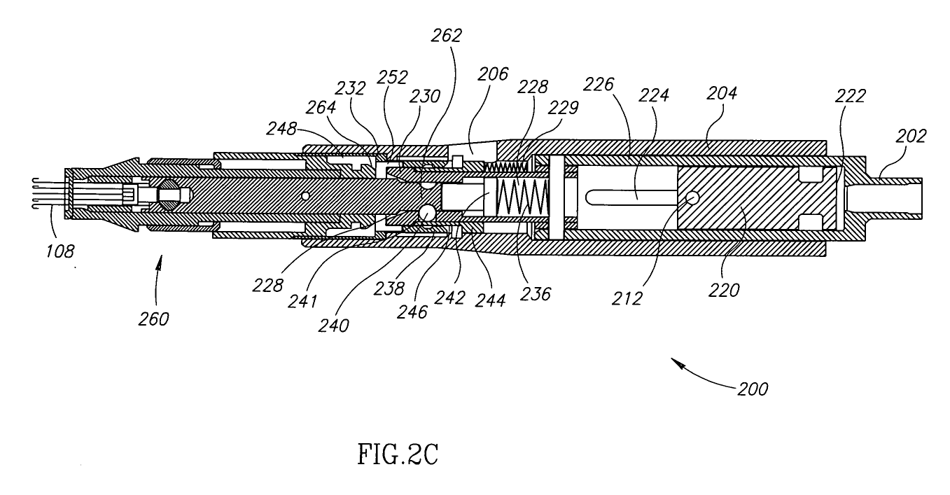

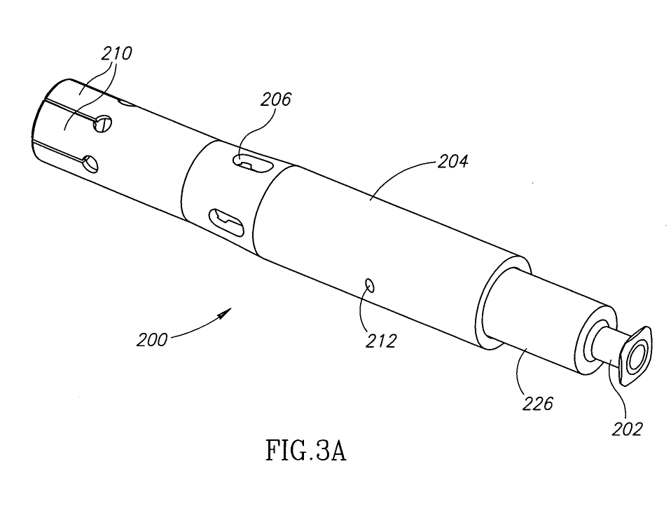

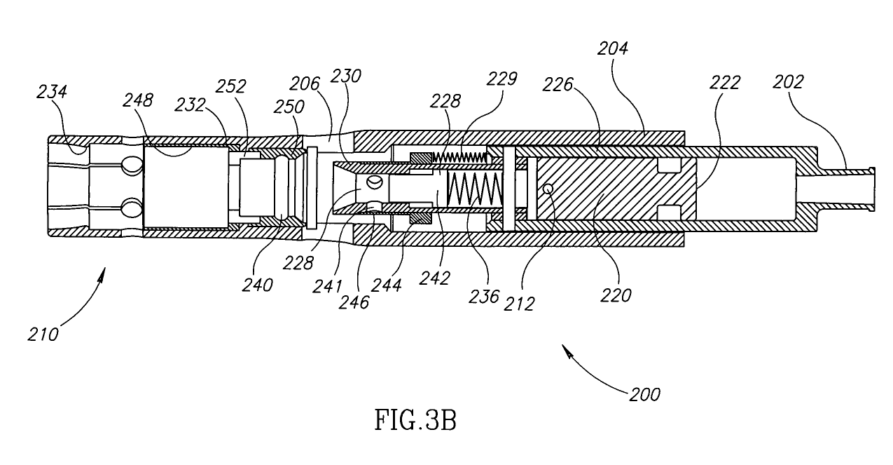

FIGS. 2A and 2B show an outside view and a cross-sectional view of a powered handle 200 in a first deployment stage and FIGS. 3A and 3B show an outside view and a cross-sectional view of powered handle 200, in a second deployment stage, in accordance with an exemplary embodiment of the invention. A capsule, for example a connector capsule or a punch capsule may be loaded into powered handle 100.

FIG. 2C shows a capsule 260 inserted into powered handle 200.

PCT publications WO 03/026475 and WO 02/30172, the disclosures of which are incorporated herein by reference, describe modular capsules for delivering anastomosis devices, which capsules can be mounted in a delivery handle. In the embodiment of the invention shown in FIGS. 2-3, powered handle 200 is used to replace the delivery system of FIG. 1, while optionally being adapted to work with other types of (e.g., mechanically actuated) capsules.

FIG. 2A is an outside view of handle 200, showing a nipple 202 (e.g., suitable for locking to a pressure hose) for attaching to a pressure source, and a body 204. At a distal end of handle 200 is a capsule holder 210 which engages a connector capsule once inserted, to prevent its falling out, for example, if the pressure is reversed and part of the capsule is pushed away from handle 200. An optional fixed pin 212 is explained below. A slot 206 is optionally provided for advanced manipulation, as described below. Handle body 204 is optionally roughened or includes ridges or finger depressions or other means to assist in holding and/or prevent slipping. In an exemplary embodiment of the invention, the stage shown in FIG. 2, is a pre-deployment stage, in which the legs of the connector 108 (FIG. 1) may be inserted into the blood vessel. Various methods of assisting such insertion may be provided, for example, a silicon ring around the legs, which ring is cut after insertion and/or after eversion, and removed using a string attached thereto. Other assisting devices are described in PCT publication WO 03/026475 and PCT application PCT/IL03/00769, published as WO 2004/028377, the disclosures of which are incorporated herein by reference.

FIG. 2B is a cross-sectional view of handle 200. FIG. 2C is a cross-sectional view of handle 200 with capsule 260 inserted therein. Fluid arriving through nipple 202 enters a chamber 222, defined by a cylinder body 226 and a piston 220. Capsule 260 fits in holder 210 and includes a pull extension 262 which is engaged by an engaging element 230. Element 230 is coupled to cylinder body 226. In operation (with reference to FIG. 3B as well), entry of fluid into chamber 222, causes relative motion between piston 220 and cylinder body 226. In an exemplary embodiment of the invention, pin 212 locks piston 220 to handle body 204 and slidebly fits in a slot 224 of cylinder body 226. Thus, as piston 220 is fixed to body 204 of handle 200, expansion of chamber 222 causes retraction of the pull extension and deployment of the connector.

Delivery capsule 260 fits inside holder 210 and is optionally engaged, for example by an optional inner ridge 234 (FIG. 2B). A base 264 of the capsule optionally rests against a step 232, when pull extension 262 fits in a receptacle 230. Movement of pull extension 262 relative to the rest of capsule 260 extends, retracts and/or tears (or otherwise deploys) the connector. In some embodiments, rotation of the pull extension is used, for example, for providing the relative motion, for a safety mechanism or for separating different stages. Optionally, a metal lining 248 is provided to hold the capsule. Optionally, a threading 250 is used to attach an extension 252 of the lining to body 204.

An exemplary mechanism for locking pull extension 262 to holder 200 is illustrated. Engagement element 230 has a lumen 228 surrounded by a plurality of balls 240, each optionally arranged opposite an aperture 241 in element 230. An inner stopper 242 in lumen 228 prevents the balls from moving inwards. Insertion of pull extension 262 pushes stopper 242 away so that the balls can enter lumen 228. A spring 236 is optionally provided to prevent inadvertent motion of stopper 242. Optionally, an outer tube 244 is used to push balls 240 into lumen 228. A spring 229 is optionally provided to urge tube 244 in the direction of balls 240. Inserting the capsule pushes element 230 backwards, while tube 244 stays in place and a forward edge 246 thereof can push balls 240 radially into lumen 228. Optionally, tube 244 is lightly frictionally engaged by body 204, so that it can also retract, as shown in FIG. 3B. In an alternative embodiment, an optional elastic ring 238 urges balls 240 inwards into lumen 228.

In an exemplary embodiment of the invention, optional slot (or other aperture) 206 is used for re-priming handle 200 after use. A screwdriver or similar tool is inserted into slot 206 and tube 244 moved away from apertures 241, so balls 240 can exit the lumen.

In operation, as shown in FIG. 3A, cylinder body 226 extends out when chamber 222 (FIG. 2B) is filled. Optionally, body 204 is held (e.g., manually), so that the extension of body 226 does not move the connector capsule 260 (FIG. 2C). Optionally, a user allows body 226 to move forward during a first extension of chamber 222, to prevent inadvertent retraction of the connector legs from aperture 106, tearing of tissue near the aperture and/or distorting of the legs of connector 108.

In embodiments where a non-tearing connector is used, the retraction of pull extension 262 can be used to retract an outer tube to release backwards legs of a connector, for example, or to pull back and/or rotate (e.g., with suitable threading) a penetration tip or cutting tube of a punch capsule.

Powered Capsule

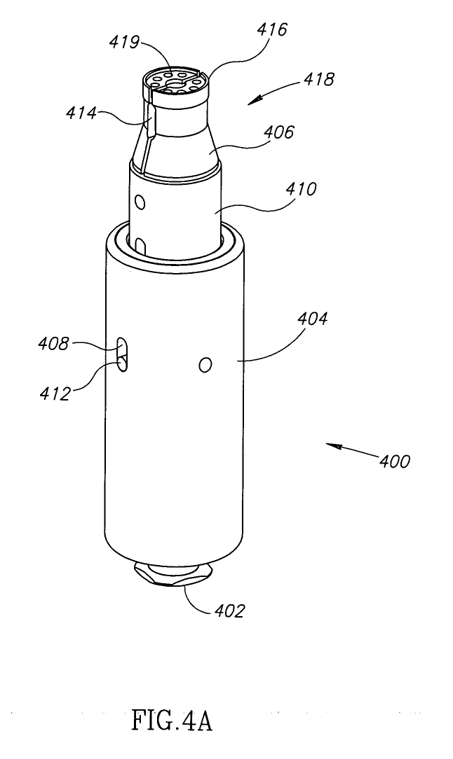

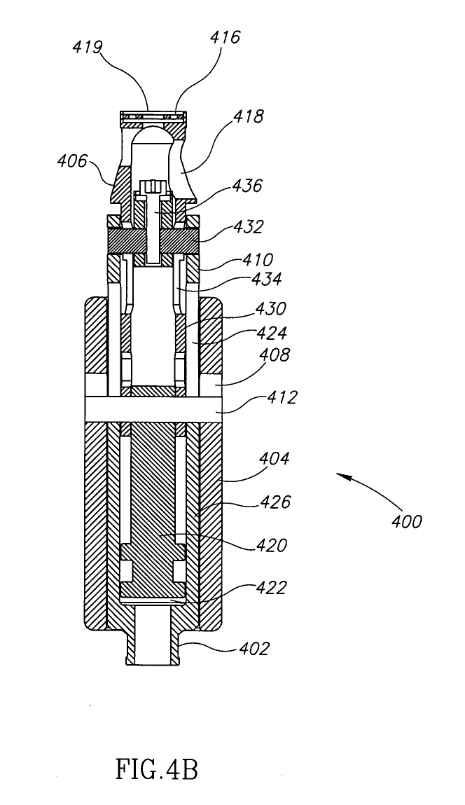

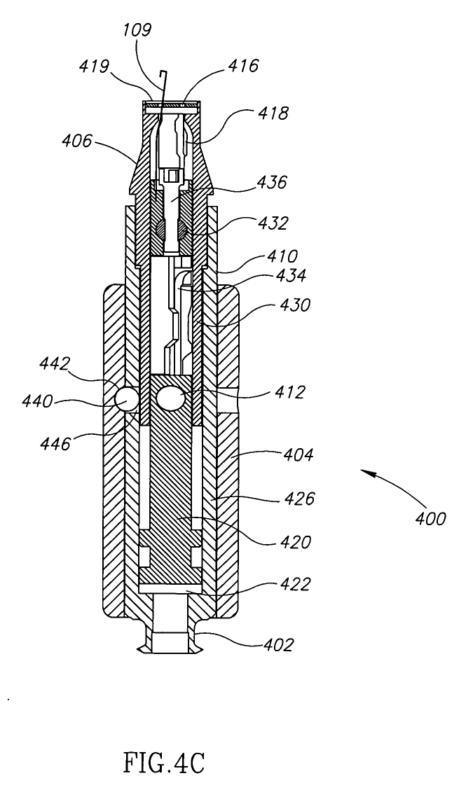

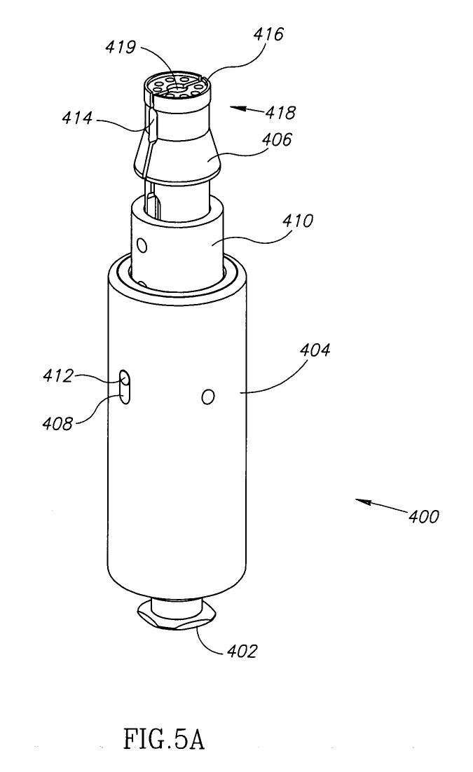

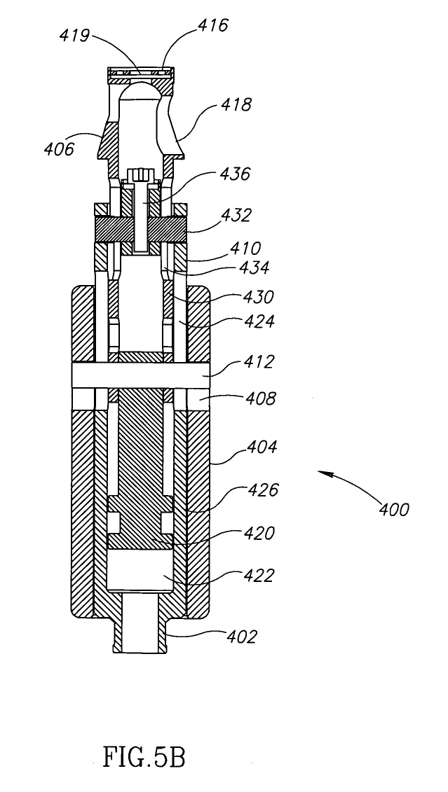

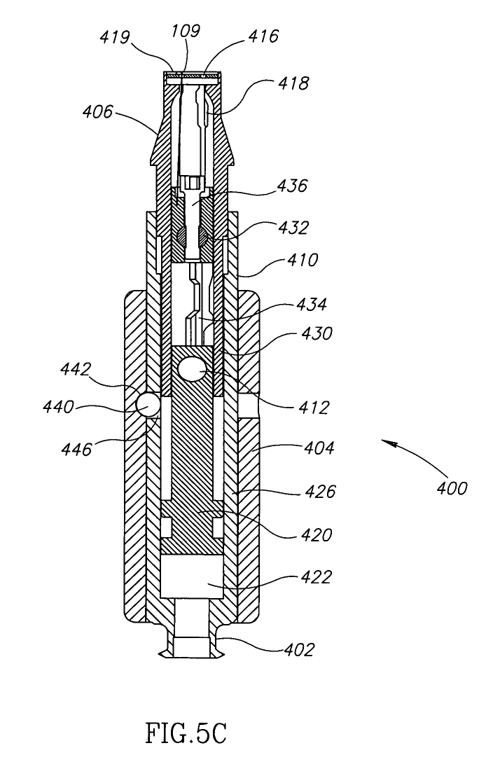

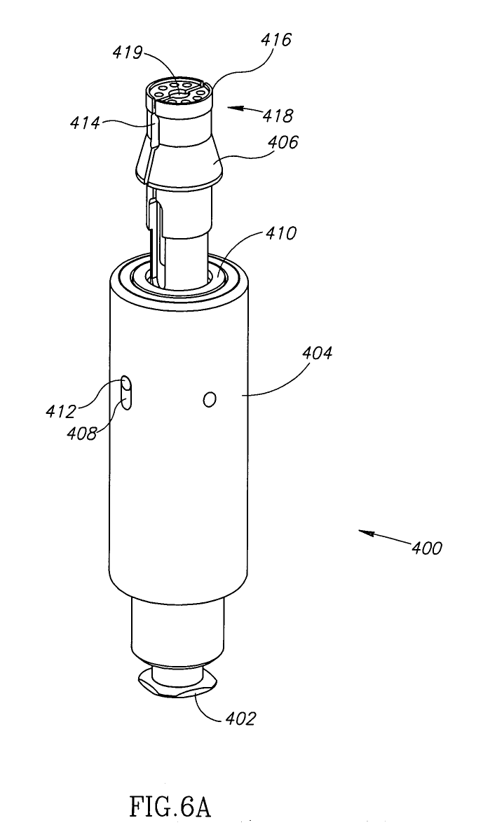

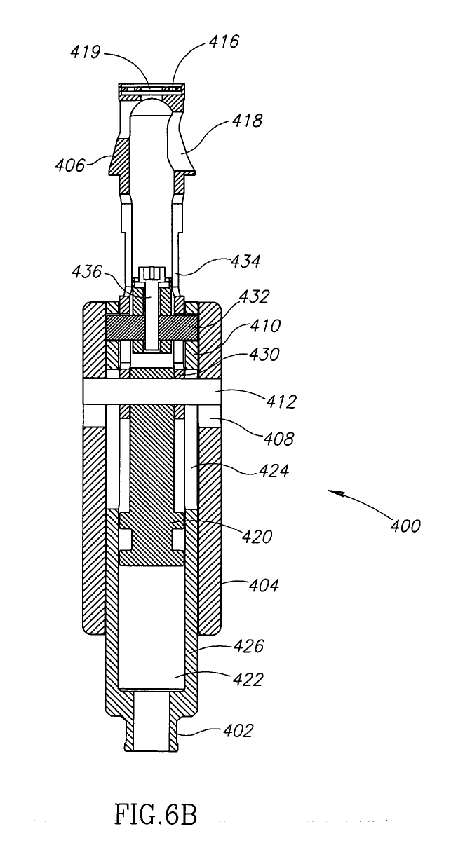

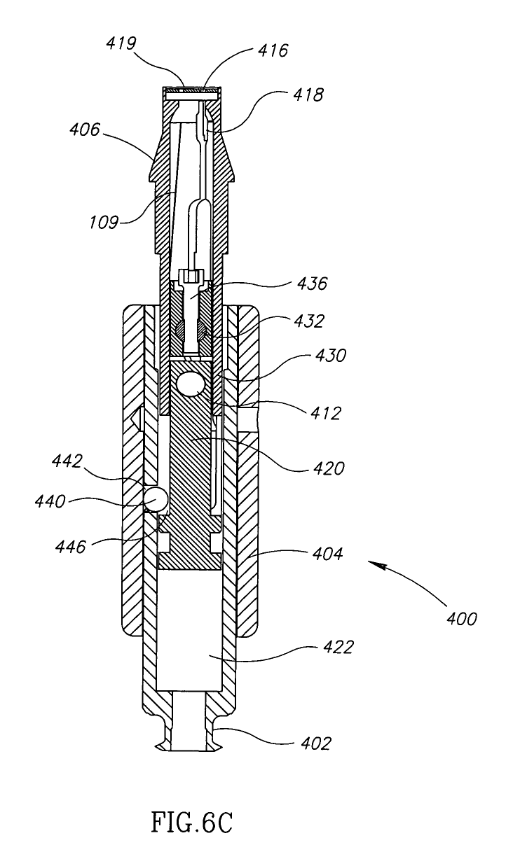

FIGS. 4A and 4B show an outside view and a cross-sectional view of a powered capsule 400, in a first deployment stage, in accordance with an exemplary embodiment of the invention. FIGS. 5A and 5B and FIGS. 6A and 6B show the capsule at second and third deployment stages. FIGS. 4C, 5C and 6C show a second side cross-sectional view, from a different direction, showing an optional ball-based selective locking mechanism.

Powered capsule 400, unlike powered handle 200 includes both a hydraulic deployment mechanism and a connector and graft holder. The connector used may be compatible with mechanical deployment systems. In the example described, a tearing connector is used. However, other connector types may be used as well.

The capsule embodiment is also used to illustrate an optional two direction of motion mechanism, which advances the connector holder towards the blood vessel, locks the connector and then retracts the connector for tearing. One advantage of this two step motion is that there may be less danger of the connector legs being inadvertently pulled out of aperture 106. Potential problems with such pulling out include, tearing of the target blood vessel, distortion of the connector and difficulty in re-inserting the legs. This type of mechanism can also be provided in a hydraulic handle.

Referring to FIG. 4A, capsule 400 comprises a nipple 402 for providing fluid, a body 404 which is optionally adapted for holding by a human operator and a connector carrying section comprising a sleeve 410, within which travels a tip 406, which is optionally splitable. A slot 414 is optionally provided in tip 406, for removal from a graft after use. As noted in the above mentioned PCT/IL03/00769 application, published as WO 2004/028377, slot 414 may be used for splitting tip 406 so that the graft can be inserted by placing it in tip 106, rather than by snaking it between apertures 418 and 419, (described next). An aperture 418 (more visible in FIG. 4B) forms an entrance for a graft, which exits from an aperture 419 at a front of tip 406. An optional connector alignment area 416 is shown, for arranging forward legs of a connector and/or for receiving medallion sections of a medallion and hook type connector, for example as described in PCT application PCT/IL03/00774, published as WO 2004/028373, the disclosure of which is incorporated herein by reference. It should be noted that other connector carrying sections can be provided for different types of connectors and for different deployment methods thereof.

Also shown in FIG. 4A is a pin 412 whose travel is limited by a slot 408 and which selectively locks tip 406 to body 404, as will be described below.

FIG. 4B shows a cross-sectional view of capsule 400. Nipple 402 is connected to a chamber 422 which is defined by a piston 420 and an outer cylinder body 426. In the embodiment shown, sleeve 410 is an extension of cylinder body 426. Tip 406 has a sleeve extension 430, which slidebly fits inside sleeve 410. Pin 412 fits in a slot 424 in sleeve 410 and locks piston 420 to sleeve extension 430.

The connector (not shown) is held by a connector holder 436, coupled to sleeve 410 by a pin 432 which fits in a slot 434 of sleeve extension 430.

In an exemplary embodiment of the invention, the various slots and pins cooperate to define relative motion between body 404, sleeve 410 and tip 406. A particular type of motion which is shown in the embodiment of FIGS. 4-6 is that of first advancing tip 406 and then retracting connector holder 436 such that the connector is locked and then torn. In some cases, the locking of the connector is provided at the end of the first motion (advancing tip 106) and the second motion only tears the connector.

Referring to all of FIGS. 4-6, the stages of deployment are now described.

FIGS. 4A-4C show an initial position, in which a graft (not shown) is inserted in aperture 418 and out of aperture 419 and optionally everted on the connector. The connector is then placed into aperture 106.

In FIGS. 5A-5B, fluid has entered chamber 422, causing its expansion. Optionally, there is some friction between body 404 and cylinder body 426, so piston 420 moves, until pin 412 reaches the end of slot 408. This motion is coupled to tip 406 which advances towards a target vessel, while the connector, coupled to holder 436, which is coupled to sleeve 410, stays back, thereby causing retraction of the connector relative to tip 406. The movement of tip 406 is optionally achieved without moving the connector relative to the target vessel, which might cause the connector to pop out of the blood vessel and/or tear the vessel.

In FIGS. 6A-6B, more fluid has entered chamber 422. However, piston 420 is constrained from forward motion by pin 412 contacting the end of slot 408. As a result, cylinder body 426 and its coupled sleeve 410 are retracted, along with connector holder 436, thereby tearing the connector and completing the deployment. As noted above, capsule 400 is optionally of a splitting type. In an exemplary embodiment of the invention, tip 406 is pre-disposed to split. Examples of such pre-disposition include, pre-stressing the tip 406, providing a spring which opens tip 406 and a peg (not shown) mounted on sleeve 410, which engages a slot in tip 406, such that retraction of sleeve 410 causes the peg to travel along the slot and widen it. Thus, in the configuration shown in FIG. 6A, tip 406 would be split apart along slot 414 (and 418).

Ball Lock Mechanism Control

FIGS. 4C, 5C and 6C show an optional embodiment in which a ball lock mechanism is used to control the order of activation instead of or in addition to a friction based mechanism. The view in these cross-sectional figures is perpendicular to that of FIGS. 4B, 5B and 6B, for example, pin 412 is illustrated end on. In the embodiment shown (e.g., FIG. 4C), one or more balls 440 are provided in one or more recesses 442 formed in body 404. Sleeve extension 430 prevents ball 440 from exiting recess 442. While in the recess, ball 440 locks recess 442 to an aperture 446 formed in cylinder body 426, thus preventing the relative motion of body 404 and cylinder body 426. In FIG. 5C, sleeve extension 430 has advanced, allowing ball 440 to exit recess 442. In FIG. 6C, ball 440 has exited recess 442 and allows body 404 to move relative to cylinder body 426. Optionally, ball 440 prevents over expansion of chamber 422 by preventing over-advancing of piston 420. Also shown in these figures is an exemplary effect on a single leg 109 of connector 108. In FIG. 4C, it is forward, in FIG. 5C, it is locked and in FIG. 6C, it is torn.

Alternative Capsule with Tabs

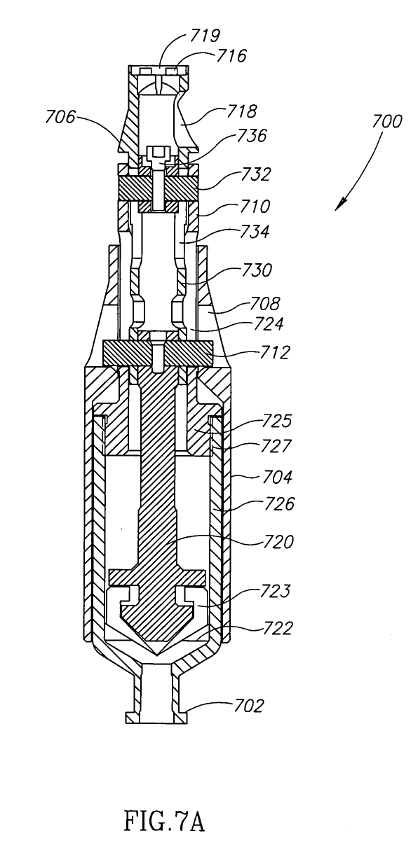

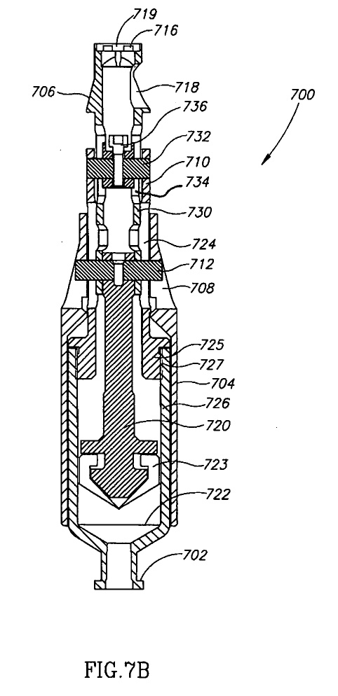

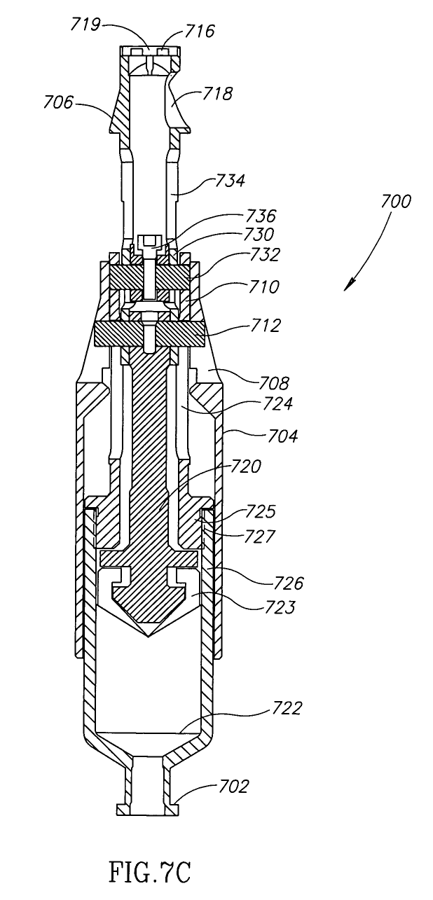

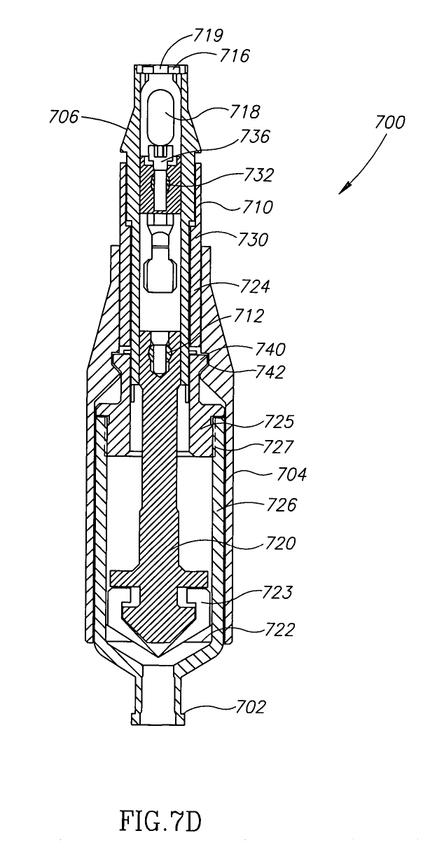

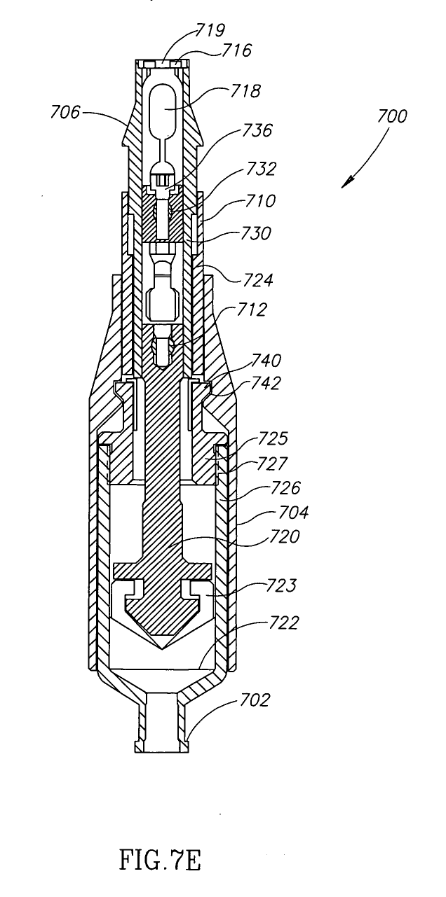

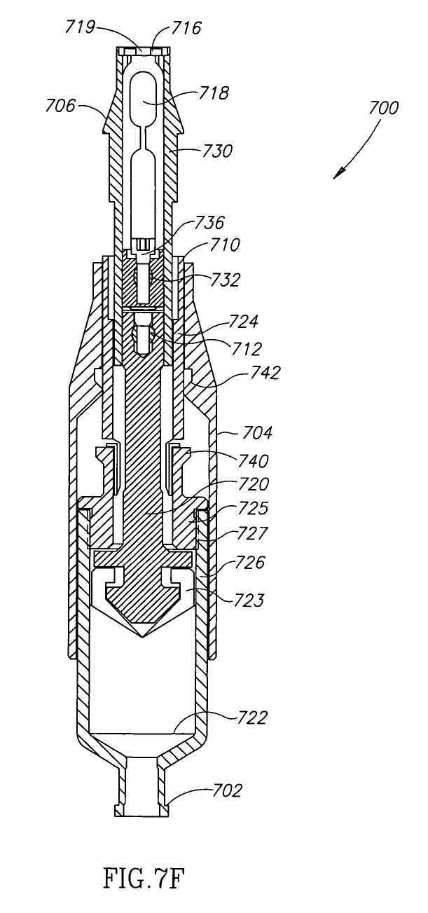

FIGS. 7A-7F are cross-sectional views of an alternative powered capsule 700, generally similar in function to capsule 400, except that the locking mechanism used to prevent motion of the cylinder body relative to the capsule body is based on plastic snap-locking, rather than on a ball lock. The reference numbers are generally corresponding between FIGS. 7A-7F and FIGS. 4-6, except for being larger by 300 (e.g., chamber 722 corresponds to chamber 422). Some elements are not described a second time.

FIGS. 7A, 7B and 7C are side cross-sectional views of capsule 700 at stages corresponding to those of FIGS. 4, 5 and 6, respectively, at a view which shows a side of a pin 712, generally corresponding in function to pin 412. Some design variations from capsule 400 are also shown, for example, the provision of a gasket 723, which optionally assists in preventing pressure leakage. Optionally, a sleeve 710 is attached to a cylinder body 726 by a base 725 of sleeve 710 being threaded to cylinder body 726 at a threaded location 727. Another optional design variation is that capsule 700 is more streamlined.

FIGS. 7D-7F show a side view of capsule 700 at the same three stages as FIGS. 7A-7C, in which the view shows pin 712 end on. The locking mechanism is clearer in this set of figures. In FIG. 7D, one or more protrusions 740 of sleeve 710 project into a recess 742, which may be, for example, in the form of an annular groove. While sloped, protrusion 740 is prevented from leaving recess 742, due to a sleeve extension 730, which fits inside of sleeve 710. In FIG. 7E, sleeve extension 730 advances away from the area of protrusions 740. In FIG. 7F, protrusions 740 snap out of recesses 742. While in the embodiment shown protrusions 740 are mounted on base 725 by elastic extensions, they may be otherwise attached.

Example System

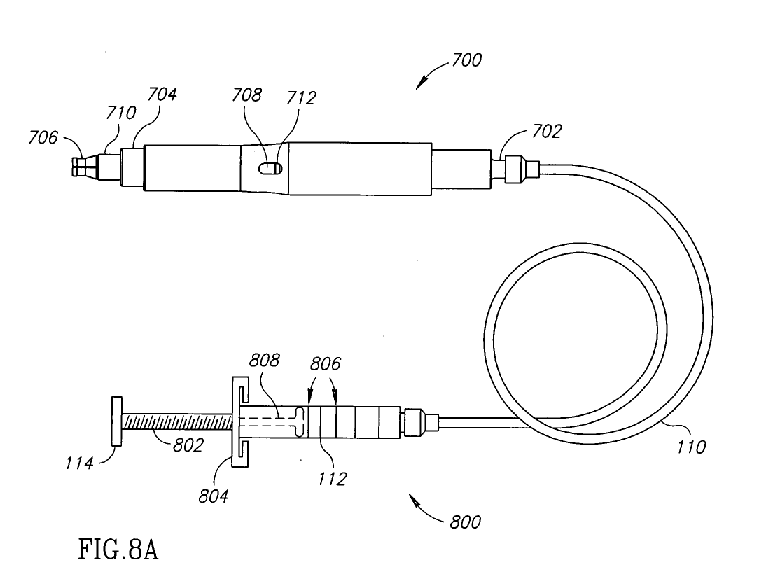

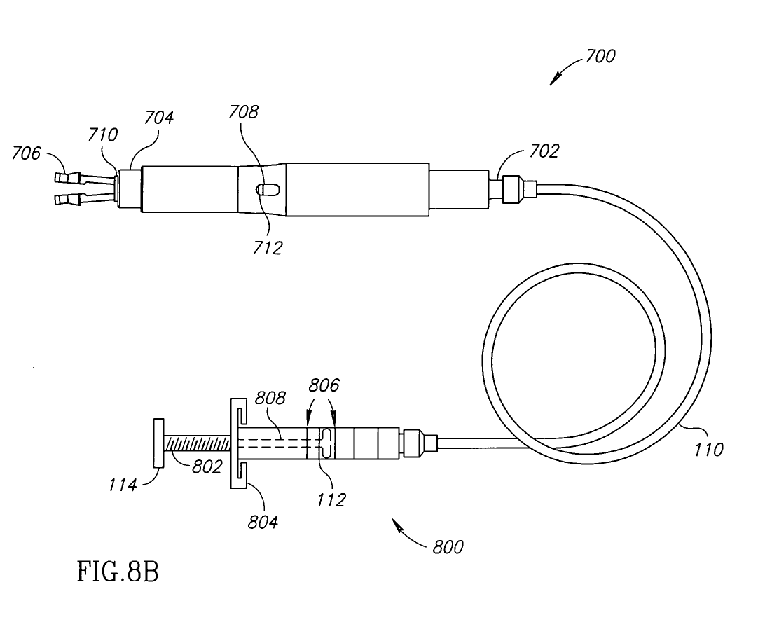

FIGS. 8A and 8B show a complete powered capsule system 800 before and after deployment, in accordance with an exemplary embodiment of the invention. FIG. 8A shows capsule 700 attached to a system as shown in FIG. 1. Piston 114 is shown as having a thread 802 which matches an inner thread of a syringe cover 804. Optional lines 806 show the position of a forward end 808 of piston 114, thereby indicating the operational stage of the system. Optionally, one or more stops are provided between the syringe and the piston, which can be removed serially to allow progression to a next step of delivery. In an alternative embodiment (referring to FIG. 7F, for example), one or more pins are positioned to interlock piston 720 and the body of capsule 700, to prevent motion, until the pins are removed. This may be useful for a pneumatic system in which the degree of advance cannot be set based on volume.

FIG. 8B shows the end of the deployment, where tip 706 is split open.

Controls

FIG. 8A shows a system where a user manipulates a syringe in order to control the anastomosis delivery. Referring back to FIG. 1, in one example of the invention a control 118 is provided on the delivery module 102 and is attached to source 112 via a cable 120. Control 118 may be, for example, a micro-switch used to advance or retract piston 114 via an electric motor (not shown) or activate a different type of pump (not shown). Alternatively, such an electrical control may be provided on source 112, for example, a finger switch. Alternatively, a separate switch, for example a foot switch, possibly with both forward and backwards direction, may be provided. Alternatively to an electrical switch, a mechanical switch (e.g., a valve) may be used to selectively admit pressurized fluid from a remote source, for example in the case of a hospital-wide compressed air source.

Control Logic

As noted above, an anastomosis delivery process can be viewed as a multi-step process. From a user's point of view, these steps are optionally delimited and optionally not. In one exemplary embodiment, once the pressure source is activated, the process continues to completion (e.g., tearing and splitting) without human intervention.

In an alternative embodiment, separate controls are provided for some or all of the stages, for example so that one control advances tip 706 and another retracts sleeve 410. In one example, a user can visually verify that all the connector legs are in place, before continuing with locking and tearing.

As noted above, in some embodiments of the invention, the control is volume based, which means that each position of the system has a corresponding volume of fluid that determines that position. In an exemplary embodiment of the invention, fine control is available using this method. For example, selective individual tearing of legs as described in PCT/IL03/00774, published as WO 2004/028373, or PCT/IL03/00770, published as WO 2004/028376 the disclosures of which are incorporated herein by reference, may be provided. As described in those applications, a connector leg can have two ends, one proximal, which is being pulled back at a pulling point and one distal, which is being held in place at a holding point. If the distance between the pulling element and the holding element is greater than the length of the leg between the pulling point and the holding point, the leg tears. Different legs can have different such lengths. Thus, as pull extension 262 (FIG. 2C) which pulls on the leg is retracted more, the distance between the pull extension and tip 206 (which holds the leg) increases. As the distance equals the length of a leg, that leg stretches a small amount and tears. By providing fine control of the distance using a hydraulic means, a user can select to tear only some of the legs, for example to allow incision shaping using the leg (e.g., as described in PCT application PCT/IL03/00769, published as WO 2004/028377). It should be noted that it may be difficult for a user to apply such fine control using a lever based or spring based mechanical lever. In a non-hydraulic embodiment, a screw is used to better control the distance, however, this may interfere with steady holding of the delivery system.

In some embodiments of the invention, a force control (e.g., based on air pressure) is provided. This may be useful, for example, to ensure that forces applied to the connector and/or tissue at certain stages in the deployment do not exceed a desired amount. In addition, this may be useful for connectors which have different deployment configurations at different applied forces (e.g., some legs tear at one force and some legs tear at another). Optionally, steps in a delivery process are set using blocking tabs which break at certain applied pressures (e.g., being arranged to block motion of the piston relative to the chamber past certain extensions) and thus allow the process to proceed to a next step.

Optionally, a damping of motion is provided, for example by providing the fluid slowly or by adding friction to the delivery mechanism. This, optionally, allows a user time to notice if something is going wrong and/or allows the user to move the delivery system if desired towards or away from the target vessel, while the pressure actuated actions are in progress.

Optionally, a ratchet mechanism is provided, so once a certain expansion of chamber 722 is reached, piston 720 cannot be retracted. In one example, the inside of cylinder 726 includes steps or hooks which engage gasket 723 if it starts moving in a reverse direction.

Optionally, a pressure release valve, for example, on tube 110, is provided for volume based embodiments, for example to limit the applied forces.

Alternative Mechanisms

Several mechanisms using hydraulic pressure for deploying tearable connectors have been described. Non-tearing connectors, in some embodiments of the invention are deployed by first pulling back the connector such that its forward legs engage the inside of a target blood vessel and then releasing backward legs. In an exemplary embodiment of the invention, the backward legs are released by retracting an overtube which kept them constrained. In an exemplary embodiment of the invention, the designs shown above are modified so that instead of pulling back the connector to tear it, such an overtube is pulled back to reveal the backwards legs of the connector. Retracting of the forward legs is optionally replaced by advancing the tip (e.g., tip 706).

Other connectors may require more complex deployment, for example, including four or more deployment stages. This can be achieved, for example, by providing another tab and sleeve mechanism as shown in FIG. 7 and/or another pin mechanism between tubes.

A rotation effect can be achieved, for example, by providing a peg on an inner (or outer) tube and a matching thread on an outer (or inner) tube, so that axial advancing also includes a component of rotation.

In the embodiment shown, nipple 702 is at an end of the capsule 700. Alternatively, it may be attached at a central portion, with, for example the piston pointed towards the proximal end rather than the connector end of the capsule. Alternatively or additionally, multiple fluid intake points are provided, for example each intake controlling a different stage of deployment.

Alternatively or additionally, both an input and an output are provided on the capsule, for example, to allow the hydraulic fluid to operate by powering a hydraulic motor, rather than a piston. Such a motor can rotate a screw which will advance and/or retract parts in the connector, for example as described in U.S. Provisional Application No. 60/505,946, the disclosure of which is incorporated herein by reference. Such a motor may also be provided for use with a pneumatic pressure source. Optionally, a pressure release valve is provided in capsule 700, for example to release over pressure of gas or liquid, for example, to prevent leakage or distortion of the capsule.

In an alternative embodiment, the hydraulic source is located in capsule 700, for example, powered by an electric motor (e.g., with an on-board battery or a power supply attached by wire).

Optionally, reducing the fluid pressure is used to retract a step in the anastomosis delivery process.

Alternative Processes

U.S. Provisional Application No. 60/505,946, describes a system which both punches a hole and deploys a connector in the hole. In this system, a punch mechanism is pulled back and to the side, to make room for the advancing of a connector. In an exemplary embodiment of the invention, that system is powered using a mechanism as described herein. Optionally, two hydraulic chambers are provided, one for retracting the punch (by expansion of a chamber in the handle, instead of advancing the handle), thereby causing a piston attached to the punch mechanism to advance proximally and retract the punch and one for deployment of the connector (using a second chamber as described herein). The two chambers may be connected, for example, by a flexible tube, to pass pressure from one chamber to the other. Optionally, pressure can flow from the first chamber to the second chamber only once the piston passes by and reveals a port from the first chamber to the second chamber. A third chamber is optionally provided (between the first and second chambers) for advancing the connector capsule into a hole made by the punch.

In an alternative implementation, two chambers are attached to a same fluid source, a first chamber having a smaller diameter and a second one having a second diameter. Thus, the motion rate of pistons in the two chambers is different. Optionally, the smaller diameter (or cross-section) chamber is used to pull back a punch section, for example using a flexible rod, which, once the punch pulled back sufficiently, urges it to one side. At the same time, the second chamber advances a connector deployment section, for example of the type described herein. A third chamber, with an even greater diameter (and optionally larger extent of motion) may be used to retract the connector section after use, for example for tearing the legs and/or approximation. Alternatively, a valve is rotated to cause the second chamber to operate in an opposite direction, with pressure being applied from an opposite side of the piston. Optionally, a plain capsule is used, which interlocks with the overtube and/or with the body. The interlocking with the overtube pulls back the overtube when the second chamber pulls back and the interlock with the body of the device tears the legs.

In an alternative embodiment, handle 200 is used to power a punching mechanism capsule (e.g., pulling back a penetration tip thereof and/or rotating a cutting tube thereof) and then the punch capsule is removed and replaced by a connector capsule. The punch capsule may leave an overtube with a valve in the punched blood vessel, to reduce leakage.

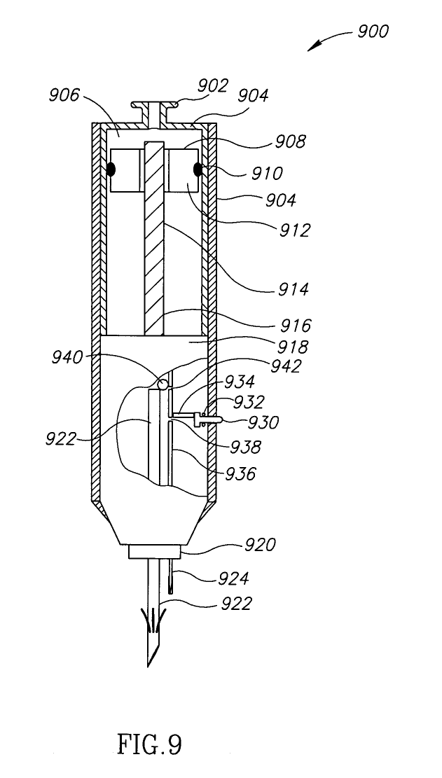

FIG. 9 shows a hydraulically actuated capsule 900, in accordance with an exemplary embodiment of the invention, illustrating the possibility of optionally features of mechanical triggering and/or rotation using a hydraulic power source. These features may be used in other embodiments as well. The rotating mechanism described below, for example, may be used in a handle-type device, to rotate a portion of a mechanical capsule.

When pressured fluid (e.g., saline or gas) enters a chamber 904 through a connector 902, it applies force against a surface 908 of a piston 912, which is thereby advanced distally. An optional gasket 910 prevents leakage against a wall 904 of the chamber. A rod 914 is coupled to piston 912 and includes a threading 916, so that advancing of piston 912 will rotate rod 914. A cutting tube 920 is optionally coupled via a base section 918 to rod 914, so that rotation of rod 914 can optionally rotate cutting tube 920. A penetration tip 922, for example as provided in the below referenced applications, optionally includes barbs. Optionally, a trigger 924 is provided, so that rotation of tube 924 and/or a retraction of penetration tip 922 are triggered by contact of the trigger with a blood vessel wall (e.g., indicating deep penetration of penetration tip 922 into a blood vessel).

In an exemplary embodiment of the invention, the following trigger/locking mechanism is used. A locking pin 930 locks base 918 against the body of capsule 900. A spring 932 tends to urge the pin out of this locking configuration. When trigger 924 moves proximally, an aperture 938 in the trigger aligns with an extension 934 of pin 930 and allows the pin top be urged out of a locking configuration.

Alternatively or additionally, a locking ball 940 is allowed to release penetration tip 922 to be spring-retracted (spring not shown), when an aperture 942 of trigger 924 is aligned with the ball. Similar and other triggering mechanism can be used as well.

Optionally, chamber 906 is elastic so that it can conform somewhat to increased fluid pressure provided therein, even if rod 914 is locked and cannot rotate.

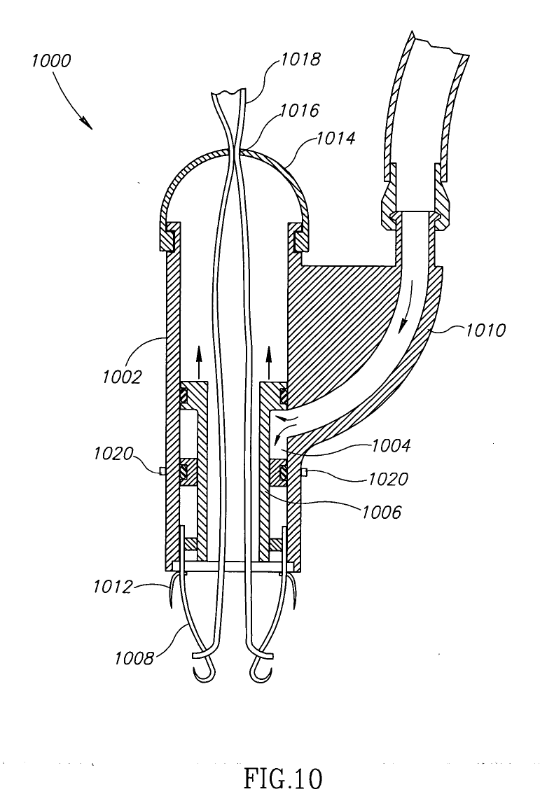

FIG. 10 shows a hollow hydraulic delivery system 1000 in accordance with an exemplary embodiment of the invention. A graft 1018 is passed through a body 1002 of system 1000. Optionally, a flexible cap 1014 has an opening 1016 to receive the graft and squeezes it gently shut, to prevent blood flow through the graft and/or through body 1002. This type of seal may also be used in non-hydraulic systems and in side mounted systems (e.g., as described above).

A connector comprising a plurality of retracting forward legs 1008 and a plurality of fixed backward legs 1012, is shown. In operation, fluid is provided into a chamber 1004, causing a piston 1006 to retract away from legs 1012, carrying legs 1008 with it. This causes engagement and locking of the connector. Further retraction can then tear legs 1012. Chamber 1004 is, for example, a ring shaped chamber or is only on one side of body 1002, for example on a side of attachment 1010 which attaches the fluid source to the body.

An optional band 1020 holds device 100 together, so that it can be easily removed after use. Body 1002 is optionally pre-split or is cut after use. Alternatively, piston 1006 meets a narrowing of an inner diameter of body 1002, so that further expansion splits body 1002. Chamber 1004 can be, for example, a slotted ring.

Applications

The above description has focused on a hand-held device. In other embodiments, the delivery system is controlled by a robot. In another embodiment, the delivery system is used for keyhole surgery. It should be noted that in an exemplary embodiment of the invention, a single hydraulic cable suffices for both power and control of the whole anastomotic deployment process, and can be used, for example, at the end of a flexible delivery tube. For example, the diameter of the system (e.g., the more forward section thereof, for example 10 cm) can be 10%, 30%, 50%, 100% or any smaller, intermediate or larger percentage than a diameter of connector to be deployed. Optionally, in an expanding connector, the diameter can be the same or smaller than a deployed connector.

Materials

The capsule may be made of any suitable material known in the art, in particular plastics. As noted above, high wear and/or high tolerance parts may be made of plastic. Alternatively or additionally, where a good seal is needed, rubber or other soft materials such as silicones may be used. In some embodiments, however, perfect sealing is not provided or intentional leaks are provided, for example to use the working fluid to keep the capsule free of blood and/or other debris or to keep the area of the anastomosis clear. Alternatively or additionally, the working fluid serves as a lubricant in the capsule or delivery handle.

It should be noted that for one time use of the device, distortion as a result of use may be allowed and/or weaker materials may be used.

Connector and Anastomosis Tools

As noted above, embodiments of the present invention may be used with various types of anastomotic connectors, anastomosis assisting tools and delivery systems. In particular, the following documents, describe connectors, delivery systems and/or other tools and methods which are useful in conjunction with embodiments of the prevent invention:
PCT/IL02/00790, filed on Sep. 25, 2002, now published as WO 03/026475;
PCT/IL02/00215, filed on Mar. 18, 2002, now published as WO 02/074188;
PCT/IL01/01019, filed on Nov. 4, 2001, now published as WO 02/47532;
PCT/IL01/00903, filed on Sep. 25, 2001 now published as WO 02/30172;
PCT/IL01/00600, filed on Jun. 28, 2001, now published as WO 02/47561;
PCT/IL01/00267, filed on Mar. 20, 2001, now published as WO 01/70091;
PCT/IL01/00266, filed on Mar. 20, 2001, now published as WO 01/70090;
PCT/IL01/00074, filed on Jan. 25, 2001, now published as WO 01/70119;
PCT/IL01/00069, filed on Jan. 24, 2001, now published as WO 01/70118;
PCT/IL00/00611, filed on Sep. 28, 2000, now published as WO 01/41624;
PCT/IL00/00609, filed on Sep. 28, 2000, now published as WO 01/41623;
PCT/IB00/00310, filed on Mar. 20, 2000, now published as WO 00/56228;
PCT/IB00/00302, filed on Mar. 20, 2000, now published as WO 00/56227;
PCT/IL99/00674, filed on Dec. 9, 1999, now published as WO 00/56223;
PCT/IL99/00670, filed on Dec. 8, 1999, now published as WO 00/56226;
PCT/IL99/00285, filed on May 30, 1999, now published as WO 99/62408; and
PCT/IL99/00284, filed on May 30, 1999, now published as WO 99/62415. The disclosures of all of these applications, which designate the US and were filed in English, are incorporated herein by reference.

In addition, the following PCT unpublished applications, all filed on Sep. 25, 2003, in English and designating the US, the disclosures of which are incorporated herein by reference describe tools and connectors which may be useful: PCT/IL03/00774, “Anastomotic Connectors”, now published as WO 2004/028373, PCT/IL03/00773, “Snare”, PCT/IL03/00770, “Sliding Surgical Clip”, now published as WO 2004/028376, PCT/IL03/00771, “Blood Vessel Cutter”, now published as WO 2004/028380 and PCT/IL03/00769, “Anastomotic Leg Arrangement”, now published as WO 2004/028377.

The following US provisional applications, the disclosures of which are incorporated herein by reference, also describe tools which may be of use, U.S. Provisional Application No. 60/492,998, filed on Aug. 7, 2003 and U.S. Provisional Application No. 60/505,946, filed on Sep. 25, 2003.

Some of these applications describe anastomosis delivery systems, hole making apparatus and/or other connectors useful in cooperation with the present invention.

While the above delivery system has been described in general for any type of blood vessel, it should be appreciated that particular modifications may be desired for certain vessel types. For example, the aorta is thicker, while a coronary vessel is thinner, thus suggesting different amounts of motion of the tip and the retracting sleeve. For example, an aorta may be 3 mm thick, while a coronary vessel may be less than 1 mm thick.

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. The exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

While the term “tube” and other geometrical shapes have been described and used for generality, it should be appreciated that this tube need not have a full body nor have a circular cross-section, in some embodiments.

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A fluid-based anastomosis system, comprising:
a body;
a fluid power source;
an actuator powered by said source; and
at least two elements coupled to said actuator and adapted to perform at least two different relative motions, responsive to serial powering of said actuator.

2. A system according to claim 1, wherein at least one of said motions comprises retraction of at least one of said elements relative to said body.

3. A system according to claim 1, wherein at least one of said motions comprises advancing of at least one of said elements away from said body.

4. A system according to claim 1, wherein a same actuator both extends and retracts a same element relative to said body, using a positive applied pressure.

5. A system according to claim 1, wherein said actuator comprises a piston-chamber mechanism that floats relative to said body.

6. A system according to claim 1, wherein said actuator selectively interlocks with said body.

7. A system according to claim 1, wherein at least one of said motions comprises rotation of at least one of said elements relative to said body.

8. A system according to claim 1, comprising a triggering mechanism which selectively unlocks at least one of said elements to move.

9. A system according to claim 1, wherein said motions are controlled by a level of pressure provided by said source.

10. A system according to claim 1, wherein said motions are controlled by volume provided by said source.

11. A system according to claim 1, wherein said actuator is a hydraulic actuator.

12. A system according to claim 1, wherein said actuator is a pneumatic actuator.

13. A system according to claim 1, wherein said body is splittable axially.

14. A system according to claim 1, comprising a stop adapted to stop a movement at a predefined amount of motion.

15. A system according to claim 1, comprising a stop adapted to stop at least one of said movements at a predefined amount of motion.

16. A system according to claim 1, wherein at least one of said elements comprises a punch.

17. A system according to claim 1, wherein at least one of said elements comprises a an anastomosis connector.

18. A system according to claim 17, wherein said motions comprise advancing and retracting said connector.

19. A system according to claim 17, wherein said motions comprise tearing said connector.

20. A system according to claim 1, wherein said body is mounted on a flexible tube.

21. A system according to claim 1, wherein said body is adapted to be held by hand.

22. A method of performing an anastomosis delivery process, comprising;
providing an anastomosis related tool at a blood vessel;
applying fluid pressure to actuate said tool; and
further applying fluid pressure to differently actuate said tool.

23. A connector delivery system, comprising:
a fluid power source;
an actuator powered by said source; and
at least two elements coupled to an anastomosis connector, which actuator moves one element relative to the other element to deploy the connector.

24. A system according to claim 23, wherein said actuator moves one element relative to the other element in at least two different movements, such that one movement retracts the connector relative to one element and the other movement releases the connector.

25. A system according to claim 23, wherein the connector is released by being tom.

26. A system according to claim 23, wherein that the pressure source controls the two movements.

27. A fluid-based anastomosis system, comprising:
a body;
a fluid power source;
an actuator powered by said source; and
at least one tool adapted to be retracted towards said body when said actuator is powered.

28. A fluid powered anastomotic system, comprising:
a fluid power source;
a handle attached to said source;
an actuator inside said handle; and
a receptacle defined in said body and adapted to receive a mechanically actuated capsule, said actuator adapted to engage a body inserted in said receptacle.

29. A fluid powered anastomotic system, comprising:
a body adapted to receive a graft vessel; and
an expanding fluid receiving chamber adjacent the graft.

30. A system according to claim 29, wherein said chamber surrounds said graft.

31. A system according to claim 29, wherein said chamber is to a side of said graft.

32. A graft delivery system comprising:
a body adapted to receive said graft; and
a flexible apertured element adapted to apply pressure to said graft and prevent blood flow therethrough.

33. A system according to claim 32, wherein said element prevents flow out of said body.
